(19) United States
(12) Patent Application Publication
(10) Pub. No.: US 2017/0164993 A1
(43) Pub. Date: Jun. 15, 2017

(54) DEVICE FOR PERFORMING THE RESECTION OF AN ORGAN

(71) Applicant: AB MEDICA, Mery-sur-Cher (FR)

(72) Inventor: Alexandre BLANC, MARSEILLE (FR)

(21) Appl. No.: 15/323,244

(22) PCT Filed: Jun. 24, 2015
(86) PCT No.: PCT/FR2015/000124

§ 371 Date: Dec. 30, 2016

(30) Foreign Application Priority Data
Jul. 6, 2014 (FR) 14/01514

(51) Int. Cl. A61B 18/08; A61B 18/10

(52) U.S. Cl. CPC A61B 18/082; A61B 18/10; A61B 2018/00077

(57) ABSTRACT

A device for resecting an organ in a cavity of a living body, in particular of a human being, includes a guide defining a duct opening out at both ends; two electrical conductors slidably mounted in the duct; an open loop made of a material that is electrically conductive and resistive in order to be suitable for producing heat by the Joule effect; elements for electrically connecting the two terminals respectively to the ends of the conductors; the loop being arranged so as to be suitable for taking on two states, namely a first state in which an orthogonal projection of its overall section onto a plane perpendicular to the longitudinal axes lies within the inside section of the duct as defined in the same plane, and a second state in which its overall section is greater than the inside section of the duct

The present invention relates to devices for resecting all or part of an organ situated in a cavity of a living body, in particular of a human being, regardless of the nature of the organ, whether healthy or unhealthy, and more particularly it relates to devices known in the field under the term “resectoscopes”.

Such a known device essentially comprises: a guide defined between a proximal end and a distal end, the guide having a through duct opening out at both ends; two electrically conductive wires that are insulated and slidably mounted in the duct, each of these two wires having first and second ends suitable for emerging, when the wires are moved in translation in the duct, respectively from the proximal and distal ends of the duct, the two first ends of the two wires having means for connection to two poles of an electrical energy source; an open loop defined between two free terminals and made of a resistive electrically conductive material in order to produce heat by the Joule effect and thus enable all or part of the determined organ to be ablated by burning; and means for electrically connecting the two free terminals of the open loop respectively to the two second ends of the two conductive wires.

Inside the duct, there are also provided channels e.g. for passing surgical instruments or the like, or introducing or insufflating various fluids into the cavity, in particular gaseous fluids, for sucking out any elements present in the cavity, etc. These means are themselves well known and are not described in greater detail herein since they do not come within the ambit of the present invention.

Resectoscopes known in the prior art have a loop of overall section, even outside the duct, that is no greater than the overall inside section of the duct, so as to be relatively easy to move in translation in the duct and so as to enter therein and exit therefrom just as easily.

Such resectoscopes are known, e.g. those described in U.S. 2009/182324, JP 2008/206994, WO 2013/064577.

In particular, U.S. 2009/182324 describes a resectoscope having at its outlet two electrical conductors that are mechanically connected together by a piece of insulating material preventing them from passing the same electric current.

However, in certain applications, in particular for ablating an organ such as a polyp, a tumor, a synechia, a malformation, or the like, in a cavity such as a uterus, once the guide has been inserted in the cavity, the cavity is expanded, e.g. by insufflating a fluid under pressure. As a result, the section of the cavity becomes greater than the outside section of the guide. Since the section of the loop is no greater than the section of the duct, it is then poorly adapted to achieve good ablation of the organ flush with its root, and it is necessary for the practitioner to apply great dexterity in order to perform such ablation, even after several manipulations.

Thus, an object of the present invention is to provide a device for resecting all or part of an organ, as defined above, in a cavity of a living body, in particular of a human being, that mitigates to a considerable extent the above-mentioned drawbacks of resectoscopes known in the prior art.

More precisely, the present invention provides a device for resecting an organ in a cavity of a living body, in particular a human being, the device comprising at least:
a guide defined between a proximal end and a distal end, said guide defining a duct opening out at both ends;
two electrical conductors, each conductor being defined along a longitudinal axis and slidably mounted in said duct, said two longitudinal axes being substantially parallel, each conductor having a first end and a second end, both suitable for emerging respectively from the proximal and distal ends of said duct, the respective first ends of the two conductors including means for connecting to at least one pole of an electrical energy source;
an open loop terminated by two free terminals, said loop being made of a material that is electrically conductive and resistive so as to be suitable for producing heat by the Joule effect; and
means for electrically connecting the two free terminals of the open loop respectively to the second ends of the two conductors;

the device being characterized by the fact that said open loop is arranged in such a manner as to be suitable for taking on two states, namely:
a first state in which an orthogonal projection of its overall section onto a plane perpendicular to the longitudinal axes lies within the inside section of said duct defined in the same plane; and
a second state in which its overall section is greater than the inside section of said duct.

Other characteristics and advantages of the present invention appear from the following description given with reference to the accompanying drawings by way of non-limiting illustration, in which:

FIG. 1 is a general diagrammatic view in perspective of a device for resecting all or part of an organ in a cavity of a living body, in particular of a human being;

FIG. 2 is an end view of an embodiment of the device of the invention;

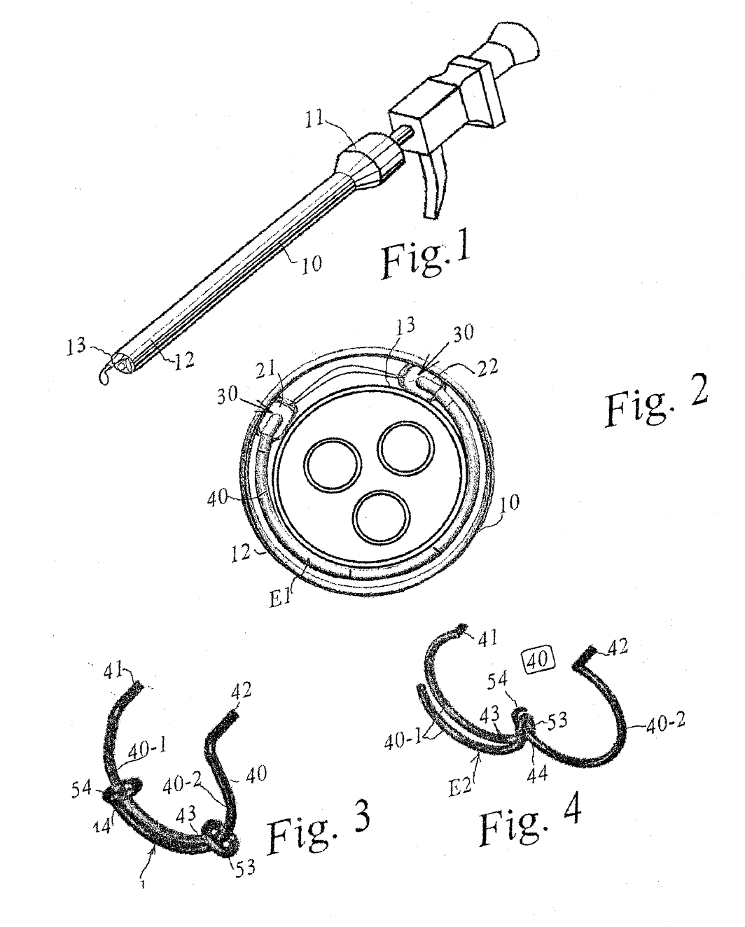

FIGS. 3 and 4 are perspective views of two embodiments of the “loop” portion of the device of the invention;

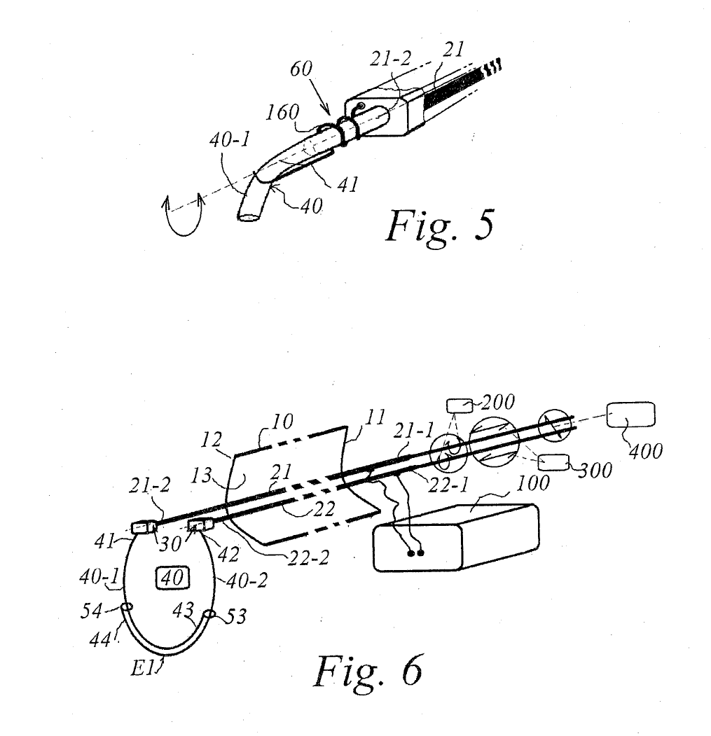

FIG. 5 is a diagrammatic perspective view showing a detail of a part of the “loop” portion of a device of the invention; and

FIG. 6 is a block diagram of the device of the invention.

It is firstly specified that, in the present description, if the adverb “substantially” is associated with a word qualifying any given means, then qualifying word should be understood as covering both its strict meaning and an approximate meaning.

The present invention relates to a device for resecting, by burning, an organ in a cavity of a living body, in particular of a human body, and it finds a particularly advantageous application in the field of hysteroscopy for intra-uterine surgery. Such a device is known to practitioners under the term “resectoscope” and it enables inter-uterine interventions to be performed in order to ablate all or part of an organ, e.g. a polyp, a tumor, a synechia, a malformation of the uterus, or the like.

With reference to the figures, the device comprises at least one guide 10 defined between a proximal end 11 and a distal end 12, the guide defining a through duct 13 that opens out at both ends, two electrical conductors 21, 22, each conductor being defined along a longitudinal axis and being slidably mounted in the duct 13, the conductors also being arranged in such a manner that their longitudinal axes are or remain substantially parallel to each other. In known manner, these conductors are also covered in electrically insulating material.

Each conductor has a first end 21-1, 22-1 and a second end 21-1, 22-2 both suitable for emerging respectively from the proximal and distal ends 11 and 12 of the duct 13 when the conductor is moved in translation in the duct. The respective first ends 21-1, 22-1 of the two conductors include means for connecting to at least one pole of an electrical energy source 100 that may be a direct current (DC) source or an alternating current (AC) source (of frequency that is adjustable, should that be necessary), for ablating the organ and then cauterizing the wound.

Specifically, in certain circumstances, and in known manner, it is possible to connect these two electrical conductors 21, 22 to a single terminal of the electrical energy source, with the other terminal then being connected by way of example to the duct 13 which is made of electrically conductive material, or even to some other conductor for certain surgical procedures.

An open loop 40 is also provided that is terminated by two free terminals 41 and 42, together with means 30 for electrically connecting the two free terminals 41 and 42 of the open loop to respective ones of the second ends 21-1, 22-2 of the two conductors 21 and 22. This open loop 40 is not electrically insulated and it is made of a material that is electrically conductive and resistive so as to be suitable for producing heat by the Joule effect up to some suitable temperature, e.g. 1000° C., so as to make it possible in known manner to ablate the determined organ by burning, optionally followed by cauterizing the wound.

According to an essential characteristic of the invention, the open loop 40 is arranged in such a manner as to be capable of taking on two states E1 and E2, namely a first state E1, shown in FIGS. 3 and 6, in which an orthogonal projection of its overall section onto a plane perpendicular to the longitudinal axes of the conductors lies within the inside section of the duct 13 as defined in the same plane, and a second state E2, shown in FIG. 4, in which its overall section is greater than the inside section of the duct 13.

In the present description, the term “section” is used essentially to define the area of a surface. Nevertheless, when the section has a well-determined shape, e.g. that of a circle or an ellipse, the term may be used exceptionally to define one of the linear dimensions of the surface, e.g. the diameter of a circle or the minor or major axis of an ellipse.

In an embodiment, the loop 40 has at least two branches 40-1 and 40-2, each branch being defined between a first end and a second end 43, 44, the respective first ends of the two branches constituting the two free terminals 41, 42, and it also has means for mounting the portions of the two branches having the second ends 43 and 44 to co-operate in such a manner that these branch portions move relative to each other while remaining continuously in contact at at least one point for ensuring electrical continuity.

In an embodiment as shown in FIG. 3, the means for mounting the portions of the two branches that include the second ends 43 and 44 in co-operation are constituted by two rings 53 and 54 located respectively at these two ends, these two branch portions passing in slidable manner respectively through the two rings.

In another embodiment as shown in FIG. 4, the means for mounting the portions of the two branches that include the second ends 43, 44 in co-operation are constituted by a ring 53 at the second end of one of the two branches, the other branch portion that includes the second end of that other branch being slidably mounted in the ring, and the second end of that other branch including an abutment 54 against which the ring 53 is suitable for coming into abutment.

More particularly, in this latter embodiment, as shown in FIG. 4, the ring 53 is formed by folding over the portion of one of the two branches that carries the second end of that branch, and the abutment 54 is constituted by a rounded bend in the portion of the other branch that includes the second end of that other branch. This embodiment presents manifest assembly advantages.

Most advantageously, the device also has means for passing the loop 40 as described above from its first state E1 (FIG. 3) to its second state E2 (FIG. 4) and vice versa. These means may be embodied in various ways.

In particular, they may be of automatic type, such as those shown functionally and diagrammatically in FIG. 5. Under such circumstances, they comprise resilient means 60 mounted to co-operate between one of the conductors and the branch that is connected to that conductor so as to cause it to pivot about an axis that coincides substantially with the longitudinal axis of the conductor.

For example, these resilient means 60 as shown in FIG. 5 are constituted by a spring 160 having a first of its ends surrounding the first end 41 of the branch 40-1 that is connected to the conductor 21, and having its other end bearing against the same branch 40-1.

When the loop 40 is inside the duct, the spring 160 is in a compressed state. In contrast, when the loop 40 is extended from the duct 13, the spring 160 relaxes and the branch 40-1 is driven resiliently to turn clockwise, this turning being obtained by elastic deformation of the branch, or possibly by means of a pivot or the like between the branch and the conductor 21.

The same may apply to the other branch 40-2, which would then be caused to turn counterclockwise.

Passing the loop 40 from its state E1 to its state E2 enables a surgical operating area and/or length to be obtained that is greater than when the loop is in its state E1, thereby facilitating the work of the practitioner.

When ablating the organ has terminated, the practitioner exerts traction on the two conductors 21, 22 relative to the guide 10 in order to return the loop 40 into the duct 13. By application of reaction forces acting in particular via the edge of the distal end 12 of the guide 10, the two branches close together, compressing the spring(s) 160, with the loop returning to its state E1 in the duct. The practitioner can then remove the guide from the cavity without difficulty.

When the loop 40 is made up of two branches as described above, the means for causing it to pass from its state E1 to its state E2, and vice versa, may be embodied in a different manner. For example, they may be constituted by means, as shown diagrammatically at 200 in FIG. 6, for causing each conductor to pivot about its respective longitudinal axis. In this embodiment, when the loop 40 is outside the duct 13, in order to cause it to pass from its state E1 to its state E2, it suffices for the practitioner to cause the two conductors to pivot in mutually opposite directions, e.g. by hand.

In order to cause the loop to pass from its state E2 to its state E1, the practitioner causes the two conductors to pivot in the opposite directions. When the loop is thus returned to its state E1, the practitioner can cause it to return into the duct 13 by pulling the conductor relative to the guide 10, prior to extracting the guide from the cavity.

In the above-described embodiments, the open loop 40 has two branches that slide over each other. Nevertheless, the open loop could be made as a single piece.

When it is made as a single piece, the means for causing the loop 40 to pass from its state E1 to its state E2, and vice versa, comprise means, as shown diagrammatically at 300 in FIG. 6, for moving the two conductors 21 and 22 in translation substantially along their respective longitudinal axes, independently of each other.

Such an embodiment is advantageous, in particular when the duct 13 is in the shape of a circular cylinder and the loop 40 is substantially in the shape of an ellipse of minor axis that is no greater than the diameter of the duct 13 and of major axis that is much greater.

Under such circumstances, when the loop 40 is in the duct 13, in its state E1, it lies in a plane that is oblique relative to the longitudinal axes of the two conductors and the axis of the duct 13. In order to cause it to pass into its state E2 after exiting the duct 13, it suffices for the practitioner to move the two conductors 21 and 22 in translation in opposite directions to each other so as to cause the loop to pivot, e.g. until it lies in a plane that is substantially perpendicular to the three above-mentioned axes.

In another embodiment that may be advantageous when the duct 13 is in the form of a circular cylinder and when the loop 40 is made as a single piece, the means for causing the loop 40 to pass from its state E1 to its state E2, and vice versa, includes means, as shown diagrammatically at 400 in FIG. 6, for moving the two conductors 21 and 22 relative to each other in a direction that is substantially perpendicular to their respective longitudinal axes, so as to move them apart from each other or towards each other. The loop passes from one of its two states to the other by deforming.

Such an embodiment is advantageous, in particular when the loop 40 in its state E1 is substantially circular in shape having a diameter that is no greater than the inside diameter of the duct 13, or having a shape that is substantially elliptical of minor axis no greater than the diameter of the duct 13, as described above, it being possible for the above-described means 200 and 400 to be combined.

It is specified that the loop 40 is made of a material that is electrically conductive and resistive, e.g. an alloy of platinum, iridium, rhodium, or stainless steel, and shaped so as to be simultaneously relatively flexible and rigid so as to be capable of being subjected to elastic and/or plastic deformations. The person skilled in the art knows how to select the solution appropriate for the surgical problem that is posed.

The way the above-described device is used can be deduced without difficulty from the description. If necessary, a better understanding may be obtained from the use of similar devices of the prior art, and use of the device is not described in greater detail herein purely for the purpose of simplifying the present description, particularly since this use does not come within the ambit of protection of the invention.

It is merely emphasized that the structure of the device of the invention as described above greatly facilitates the work of the practitioner, particularly but not exclusively when the cavity in which the device is to be used for ablating an organ by burning e.g. a uterine cavity, presents, as explained in the introduction of the present description, a portion having a section that is much greater than the section of its entrance, for whatever reason.

1. A device for resecting an organ in a cavity of a living body, in particular a human being, the device comprising at least:
a guide (10) defined between a proximal end (11) and a distal end (12), said guide defining a duct (13) opening out at both ends;
two electrical conductors (21, 22), each conductor being defined along a longitudinal axis and slidably mounted in said duct, said two longitudinal axes being substantially parallel, each conductor having a first end (21-1, 22-1) and a second end (21-2, 22-2), both suitable for emerging respectively from the proximal and distal ends (11, 12) of said duct (13), the respective first ends (21-1, 22-1) of the two conductors including means for connecting to at least one pole of an electrical energy source (100);
an open loop (40) terminated by two free terminals (41, 42), said loop being made of a material that is electrically conductive and resistive so as to be suitable for producing heat by the Joule effect; and
means (30) for electrically connecting the two free terminals (41, 42) of the open loop respectively to the second ends (21-2, 22-2) of the two conductors (21, 22);
wherein said open loop (40) is arranged in such a manner as to be suitable for taking on two states (E1, E2), namely:
a first state (E1) in which an orthogonal projection of its overall section onto a plane perpendicular to the longitudinal axes lies within the inside section of said duct (13) defined in the same plane; and
a second state (E2) in which its overall section is greater than the inside section of said duct (13).

2. A device according to claim 1, wherein said loop (40) has at least two branches (40-1, 40-2), each branch being defined between a first end and a second end (43, 44), said respective first ends of the two branches constituting the two free terminals (41, 42), and means for mounting the portions of the two branches that have the second ends (43, 44) in co-operation in such a manner that these branch portions move relative to each other while remaining in contact at at least one point.

3. A device according to claim 2, wherein the means for mounting the portions of the two branches that include the second ends (43, 44) in co-operation are constituted by two rings (53, 54) located respectively at said two second ends, said two branch portions passing in slidable manner respectively through the two rings.

4. A device according to claim 2, wherein the means for mounting the portions of the two branches that include the second ends (43, 44) in co-operation are constituted by a ring (53) located at the second end of one of the two branches, the portion of the other branch that includes the second end of that other branch being slidably mounted in said ring, and the second end of said other branch including an abutment (54) against which said ring (53) is suitable for coming into abutment.

5. A device according to claim 4, wherein said ring (53) is formed by folding over the portion of one of the two branches carrying the second end of that branch, and that said abutment (54) is constituted by a rounded bend in the portion of the other branch that includes the second end of that other branch.

6. A device according to claim 1, further comprising means for causing said loop (40) to pass from its first state (E1) to its second state (E2), and vice versa.

7. A device according to claim 6, wherein the means for causing said loop (40) to pass from its first state (E1) to its second state (E2) and vice versa comprise resilient means (60) mounted in cooperation with at least one of the two branches (40-1, 40-2) so as to cause it to pivot about an axis that substantially coincides with the longitudinal axis of the conductor (21, 22) to which it is connected.

8. A device according to claim 6, wherein the means for causing said loop (40) to pass from its first state (E1) to its second state (E2) and vice versa comprise means (200) for causing each conductor to pivot about its respective longitudinal axis.

9. A device according to claim 6, wherein said loop (40) is formed as a single piece, and the means for causing said loop (40) to pass from its first state (E1) to its second state (E2) and vice versa comprise means (300) for moving the two conductors (21, 22) in translation substantially along their respective longitudinal axes, independently of each other.

10. A device according to claim 9, wherein said duct (13) is circularly cylindrical in shape, said loop (40) having a shape that is substantially an ellipse of minor axis that is not greater than the diameter of said duct (13).

11. A device according to claim 6, wherein said loop (40) is formed as a single piece, and the means for causing said loop (40) to pass from its first state (E1) to its second state (E2) and vice versa comprise means (400) for moving the two conductors (21, 22) relative to each other in a direction substantially perpendicular to their respective longitudinal axes, so as to move them towards each other or apart from each other.

12. A device according to claim 11, wherein said duct (13) is circularly cylindrical in shape, and said loop (40) in its first state (E1) presents a shape that is substantially circular of diameter that is no greater than the diameter of said duct (13).
